SEQUENCE LISTING (30 sequences)

SEQ ID NO: 1 - protein, 4 aa, Artificial Sequence
  Substrate
  VARIANT 2: Xaa = tetrahydro-isoquinoline-3-carboxylic acid
YXSD

SEQ ID NO: 2 - protein, 4 aa, Artificial Sequence
  Substrate
  VARIANT 2: Xaa = tetrahydro-isoquinoline-3-carboxylic acid
RXSD

SEQ ID NO: 3 - protein, 4 aa, Artificial Sequence
  Substrate
  VARIANT 1: Xaa = diaminopimelic acid (Dap)
  VARIANT 2: Xaa = Ornithine (Orn)
  VARIANT 3: Xaa = Phe(3-Cl)
  VARIANT 4: Xaa = Cys(MeOBzl)
XXXX

SEQ ID NO: 4 - protein, 4 aa, Artificial Sequence
  Substrate
  VARIANT 1: Xaa = Diaminopimelic acid (Dap)
  VARIANT 2: Xaa = Ornithine (Orn)
  VARIANT 3: Xaa = Phe(3-Cl)
  VARIANT 4: Xaa = Nle(OBzl)
XXXX

SEQ ID NO: 5 - protein, 4 aa, Artificial Sequence
  Substrate
HRFR

SEQ ID NO: 6 - protein, 8 aa, Artificial Sequence
  Substrate
TGRGPSWV

SEQ ID NO: 7 - protein, 7 aa, Artificial Sequence
  Substrate
GSGRSAG

SEQ ID NO: 8 - protein, 11 aa, Artificial Sequence
  Substrate
GGSGRSANAKC

SEQ ID NO: 9 - protein, 13 aa, Artificial Sequence
  Substrate
LGGSGRSANAILE

SEQ ID NO: 10 - protein, 8 aa, Artificial Sequence
  Substrate
LSGRSDNH

SEQ ID NO: 11 - protein, 7 aa, Artificial Sequence
  Substrate
LSGRDNH

SEQ ID NO: 12 - protein, 4 aa, Artificial Sequence
  Substrate
GFLG

SEQ ID NO: 13 - protein, 4 aa, Artificial Sequence
  Substrate
FRRG

SEQ ID NO: 14 - protein, 5 aa, Artificial Sequence
  Substrate
GPLGV

SEQ ID NO: 15 - protein, 7 aa, Artificial Sequence
  Substrate
GPLGVRG

SEQ ID NO: 16 - protein, 6 aa, Artificial Sequence
  Substrate
PVGLIG

SEQ ID NO: 17 - protein, 6 aa, Artificial Sequence
  Substrate
  VARIANT 2: Xaa = citrulline (Cit)
  VARIANT 4: Xaa = homophenylalanine (Hof)
PXGXYL

SEQ ID NO: 18 - protein, 7 aa, Artificial Sequence
  Substrate
GPLGLAG

SEQ ID NO: 19 - protein, 8 aa, Artificial Sequence
  Substrate
GPLGVAGL

SEQ ID NO: 20 - protein, 8 aa, Artificial Sequence
  Substrate
GPLGIAGQ

SEQ ID NO: 21 - protein, 8 aa, Artificial Sequence
  Substrate
GPVGLIGK

SEQ ID NO: 22 - protein, 4 aa, Artificial Sequence
  Substrate
PLGL

SEQ ID NO: 23 - protein, 10 aa, Artificial Sequence
  Substrate
SLAPLGLQRR

SEQ ID NO: 24 - protein, 12 aa, Artificial Sequence
  Substrate
GRIGFLRTAKGG

SEQ ID NO: 25 - protein, 7 aa, Artificial Sequence
  antibody sequence
  VARIANT 2, 6: Xaa = any amino acid
PXTFGXG

SEQ ID NO: 26 - protein, 7 aa, Artificial Sequence
  antibody sequence
  VARIANT 1, 2, 5, 7: Xaa = any amino acid
XXWVXQX

SEQ ID NO: 27 - protein, 7 aa, Artificial Sequence
  antibody sequence
  VARIANT 1, 3, 6: Xaa = any amino acid
XDXWGXG

SEQ ID NO: 28 - protein, 109 aa, Artificial Sequence
  antibody sequence
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKR

SEQ ID NO: 29 - protein, 118 aa, Artificial Sequence
  antibody sequence
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 30 - protein, 29 aa, Artificial Sequence
  linker sequence
GGGGSGGGGSGGGGSGGGGSGGGGSGGGG

(19) United States
(12) Patent Application Publication
(10) Pub. No.: US 2024/0156974 A1
(43) Pub. Date: May 16, 2024

(54) COMPOSITIONS AND METHODS FOR MODULATION OF ANTIBODY ACTIVITY

(71) Applicants: Institute For Cancer Research D/B/A Tha Research Institute Of Fox Chase Cancer Center, Philadephia, PA (US); The Wister Institute Of Anatomy And Biology, Philadephia, PA (US)

(72) Inventors: John KARANICOLAS, Philadephia, PA (US); Jittasak Khowsathit, Pathum Thani (TH); Joseph Salvino, Philadelphia, PA (US); Daniel Pushparaju Yeggoni, Philadelphia, PA (US); Sven Miller, Philadelphia, PA (US)

(21) Appl. No.: 18/282,432

(22) PCT Filed: Mar. 17, 2022
(86) PCT No.: PCT/US2022/020683

§ 371 Date: Sep. 15, 2023

Related U.S. Application Data
(60) Provisional application No. 63/162,208, filed on Mar. 17, 2021.

(51) Int. Cl. A61K 47/65; A61K 47/54

(52) U.S. Cl. CPC A61K 47/65; A61K 47/542

(57) ABSTRACT

Compositions and methods for modulating antibody activity are disclosed.

This application claims priority under 35 U.S.C. § 119(e) to U.S. Provisional Patent Application No. 63/162,208, filed on Mar. 17, 2021. The foregoing application is incorporated by reference herein.

### FIELD OF THE INVENTION

The present invention relates to the field of immunology. More specifically, the invention provides compositions and methods for modulating antibody activity.

### BACKGROUND OF THE INVENTION

Several publications and patent documents are cited throughout the specification in order to describe the state of the art to which this invention pertains. Each of these citations is incorporated herein by reference as though set forth in full.

Antibody-derived therapeutics have proved to be very effective in several disease conditions where conventional therapies have failed and several therapeutic antibodies have gained clinical use for major diseases including various cancers, chronic inflammatory diseases, autoimmune diseases, and infection. However, since therapeutic antibodies are typically administered in the blood circulation in large doses and target molecules that have additional functions unrelated to the disease, such systemic inhibition of the target can produce undesirable side effects. For example, patients receiving rituximab therapy for B-cell lymphoma have increased frequency of bacterial infections (Ram, et al. (2009) Leukemia Lymphoma 50(7):1083-95). Similarly, antibody-mediated targeting of tumor necrosis factor (TNF) (e.g., with adalimumab, infliximab, golimumab, or certolizumab), which is implicated in conditions such as rheumatoid arthritis, Crohn's disease, and ankylosing spondylitis, often escalates susceptibility to opportunistic pathogens and reactivation of previously acquired infections such as Mycobacterium tuberculosis (Zelova, et al. (2013) Inflamm. Res., 62:641-51; Siebert, et al. (2015) Pharmacol. Rev., 67:280-309; Selmi, et al. (2014) Immunol Res., 60:277-88; Murdaca, et al. (2015) Expert Opin. Drug Saf., 14:571-82; Wallis, R. S. (2009) Curr. Opin. Infect. Dis., 22(4):403-9; Martin-Mola, et al. (2009) Rheum. Dis. Clin. North Am., 35(1):183-99).

Immune-checkpoint inhibitors have completely transformed the treatment landscape for many patients with advanced cancers. For example, the dramatic and unprecedented patient responses to anti-cytotoxic T-lymphocyte-associated protein 4 (CTLA-4) antibody ipilimumab for late-stage (metastatic) melanoma spawned clinical trials in additional cancers, as well as in combinations with the anti-programmed cell death protein 1 (PD-1) and anti- programmed death-ligand 1 (PD-L1) antibodies. However, the profound efficacy of immune-checkpoint inhibitors seems inexorably linked to their shared limitation: extreme toxicity that prevents many patients from even completing a course of treatment. Indeed, immune-checkpoint inhibitors are also associated with severe, and sometimes fatal, adverse side effects, largely due to the non-specific activation of T-cells (Winer, et al. (2018) J. Thorac. Dis., 10:S480-S489; Myers, et al. (2018) Curr. Oncol., 25:342-347; Johnson, et al. (2015) Ther. Adv. Med. Oncol., 7:97-106; Cappelli, et al. (2017) Rheum. Dis. Clin. North. Am., 43:65-78; Topalian, et al. (2015) Cancer Cell 27:450-461).

Managing adverse effects such as immune-related adverse events thus represents the single primary hurdle to broader applicability of these valuable therapies. Clearly, improved control of the activity of therapeutics antibodies, thereby reducing unwanted side effects, are needed.

### SUMMARY OF THE INVENTION

In accordance with the present invention, prodrugs for activating allosteric antibodies are provided. In a particular embodiment, the prodrug comprises an effector molecule and a protecting group which comprises a substrate for an activating or cleaving enzyme or protease and a self-immolating linker. Generally, the activating or cleaving protease or enzyme is specific to the tumor microenvironment or upregulated in the tumor microenvironment. In a particular embodiment, the activating enzyme is a protease.

In accordance with another aspect of the instant invention, methods of modulating the activity of an allosteric antibody are provided, particularly in the context of therapeutic treatment of a subject. Generally, the method comprises contacting the allosteric antibody with a prodrug, thereby restoring and/or increasing the antigen-binding activity of the allosteric antibody. In a particular embodiment, the method comprises administering the allosteric antibody to a subject in need thereof and administering a prodrug at the desired time and location for activity of the allosteric antibody.

### BRIEF DESCRIPTION OF THE DRAWINGS

FIG. 1A provides a schematic for the synthesis of a prodrug comprising Stitch3 (6-phenylmethoxy-1H-indazole) and a cathepsin B-responsive, self-immolating linker. FIG. 1B provides a schematic of the activation of the prodrug. FIG. 1C provides the results of a mass spectrometry analysis showing the conversion of prodrug (730 Da) to Stitch3 (225 Da). FIG. 1D provides examples of prodrugs comprising a substrate for β-glucuronidase and DT-diaphorase (NQO1). Replacement of β-gluc with β-gal yields a prodrug for β-galactosidase.

FIG. 2 provides a graph showing the results of a CTLA-4/CD80 homogeneous time resolved fluorescence (HTRF) binding assay. The assay was performed with wild-type antibody (WT; an anti-CTLA-4 scFv construct based on ipilimumab) or a triple-mutant antibody (Rip3; the WT scFv construct with the triple mutation (V_{L}F98G/V_{H}V37A/V_{H}W110G)), optionally in the presence of Stitch3, prodrug (Prodrug1), or prodrug and cathepsin B (CatB).

### DETAILED DESCRIPTION OF THE INVENTION

Adverse effects such as immune-related adverse events are spatially distinct from the site of therapeutic efficacy for therapeutic antibodies. For example, in the case of ipilimumab, its anti-cancer effects are due to activity in the tumor microenvironment whereas its immune-related adverse events are traced to its engagement of CTLA-4 in the lymphoid organs. By eliminating “on-target off-tumor” antigen binding, ipilimumab's therapeutic benefits can be realized without its associated toxicities. In other words, a “tumor-selective” variant of ipilimumab will increase therapeutic benefits while decreasing or eliminating undesirable side effects and toxicities.

To achieve the above desired goals, the instant invention describes allosteric antibodies and prodrugs of effector molecules which 1) bind the allosteric antibody and increase or restore the binding activity of the allosteric antibody, and 2) have tumor environment selectivity. Effector molecules which fill internal cavities formed from mutations in proteins such as allosteric antibodies do not have inherent tumor selectivity. However, the designed prodrugs of the instant invention afford the desired tumor selectivity.

Effector molecules such as JK43 (also known as Stitch3) bind to a fully-enclosed site of the allosteric antibody. As such, modest or even minor perturbations to the shape can completely abrogate rescue of the allosteric antibody (Khowsathit, et al., ACS Cent Sci. (2020) 6:390-403). Therefore, the addition of permanent targeting ligands to the effector molecules in order to direct the effector molecules to the tumor environment is unlikely to work.

Herein, prodrug effector molecules are provided which are tumor selective and/or specific. The prodrugs comprise the effector molecule and a protecting group. The addition of the protecting group prevents the prodrug from fitting into the designed cavity of the allosteric antibody, thereby preventing the effector molecule from rescuing the activity of the allosteric antibody. However, in the tumor microenvironment, the protecting group will be released from the prodrug to reveal the effector molecule, which can then bind the allosteric antibody and increase or restore the binding activity of the allosteric antibody. This strategy will produce an abundance of effector molecule selectively at the tumor site which, in turn, will direct localized antigen-binding by the rescued allosteric antibody.

As stated hereinabove, the prodrugs of the instant invention comprise an effector molecule and a protecting group. In a particular embodiment, the protecting group of the instant invention comprises a linker, particularly a self-immolating linker. For example, the prodrug may comprise the effector molecule covalently attached to a protecting group wherein the protecting group comprises a linker, particularly a self-immolating linker, and a site/substrate recognized and/or cleaved by a tumor specific, activating or cleaving enzyme or protease.

A self-immolating linker (also known as a self-immolative linker) is a linker that upon some activation event (e.g., cleavage from another group (e.g., an enzyme substrate or chemical or pH trigger, typically enzymatic cleavage)) results in a reaction (typically a cascade of reactions) which results in the removal of the linker from another group (e.g., effector molecule), leaving no residual atoms on the other group (e.g., effector molecule). In a particular embodiment, self-immolating linker has a fragmentation process which is either 1,4-, 1,6-, or 1,8-elimination or cyclization. Self-immolating linkers have been described (Tranoy-Opalinski, et al. (2008) Anticancer Agents Med. Chem. (2008) 8:618-37; Alouane, et al. (2015) Angew Chem. Int. Ed. Engl., 54:7492-509; Dubowchik, et al. (1998) Bioorg. Med. Chem. Lett., 8:3341-6; Karnthaler-Benbakka, et al. (2019) Chem. Biodivers., 16:e1800520; Younes, et al. (2010) N. Engl. J. Med. (2010) 363:1812-21; Gonzaga, et sl. (J. Pharm. Sci. (2020) 109:3262-3281); Sengee et al. (Bioconjugate Chem. (2019) 30:1489-1499); each incorporated by reference herein). In certain embodiments, the self-immolating linker comprises a heterobifunctional linker (e.g., a heterobifunctional linker comprising a thiol-reactive group at one end and a hydroxyl- or amine-reactive group at the other (e.g., leading to the linker —CO—X—CR¹—(CH₂)n-CR²R³—S—, wherein X=NR or O, n=0-4, and R, R¹, R², R³=H or alkyl (e.g., C1-C4, particularly methyl)); see Sengee et al. (Bioconjugate Chem. (2019) 30:1489-1499)). In certain embodiments, the self-immolating linker comprises a p-aminobenzyl unit (e.g., p-aminobenzyloxycarbonyl (PAB)). For example, a p-aminobenzyl alcohol can be attached to an amino acid unit (e.g., via an amide bond) and a bond (e.g., carbamate, methylcarbamate, or carbonate) is made between the alcohol and the effector molecule. Examples of self-immolating linkers can comprise, without limitation: a p-aminobenzyl unit (e.g., p-aminobenzyloxycarbonyl (PAB)), 2-aminoimidazol-5-methanol derivatives (U.S. Pat. No. 7,375,078; Hay et al. (1999) Bioorg. Med. Chem. Lett., 9:2237; incorporated herein by reference) and ortho- or para-aminobenzylacetals.

Self-immolating linkers confer several advantages. First, self-immolating linkers provide spatial separation between the effector molecule and the functional group recognized by the cleaving enzyme. This allows different effector molecules to be incorporated into the prodrug without effecting enzymatic cleavage and activation. Second, the use of a linker separates the chemistry of attaching the protecting group from the effector molecule. In other words, different the functional groups recognized by a cleaving enzyme can be attached to the linker regardless of the effector molecule. In essence, this conceptually separates the protecting group from the effector molecule so that the two can be separately optimized in a modular fashion.

Many proteases are up-regulated in the tumor microenvironment (Vasiljeva, et al. (2019) Biol Chem., 400(8): 965-977; Weidle, et al. (2014) Cancer Genomics Proteomics, 11:67-79; Vandooren, et al. (2016) Adv. Drug Deliv. Rev., 97:144-55). Inasmuch as the prodrugs of the instant invention are synthetic and not genetically-encoded, non-natural amino acids and/or alternate linkages may be used in place of peptide bonds. Non-peptidic or non-natural sequences can provide highly selective substrates that are not possible using natural amino acid sequences (Wood, et al. (2005) J. Am. Chem. Soc., 127:15521-7; Kasperkiewicz, et al. (2017) FEBS J., 284:1518-39; Poreba, et al. (2014) Cell Death Differ., 21:1482-92; Poreba, et al. (2018) Chem. Sci., 9:2113-29; Dubowchik, et al. (1998) Bioorg. Med. Chem. Lett., 8:3341-6).

As explained herein, the protecting groups of the instant invention comprise a functional group recognized by a cleaving enzyme (e.g., protease). The functional group is typically a substrate of the cleaving enzyme. Cleavage of the functional group or substrate typically occurs adjacent to the self-immolating linker, thereby causing activation of the self-immolating linker and removal of the self-immolating linker from the effector molecule. The activating (e.g., cleaving) enzymes or proteases are preferably specific to the tumor and/or its microenvironment and/or are upregulated in the tumor and/or its microenvironment. In other words, the activating (e.g., cleaving) enzymes or proteases are expressed only or almost exclusively in the tumor microenvironment compared to normal (non-cancerous) tissue (e.g., tumor specific) or is expressed at higher levels (e.g., 2-fold or higher) in the tumor microenvironment compared to normal (non-cancerous) tissue (e.g., up-regulated). Examples of tumor specific or tumor up-regulated enzymes and proteases encompassed by the instant invention include, without limitation: cathepsin B, legumain, cathepsin L, matrix metalloproteinase 2 (MMP2), MMP9, MMP14, matriptase, urokinase-type plasminogen activator (uPA), β-glucuronidase, β-galactosidase, neutrophil-secreted elastase (NSE), and DT-diaphorase (NQO1) (Vandooren J, Opdenakker G, Loadman P M, Edwards D R. Proteases in cancer drug delivery. Adv Drug Deliv Rev. 2016; 97:144-55; Legigan, et al. (2012) J. Med. Chem., 55:4516-20; Renoux, et al. (2017) Chem. Sci., 8:3427-33; Legigan, et al. (2012) Angew Chem. Int. Ed. (2012) 51:11606-10; Sharma, et al. (2018) Biomaterials 155:145-51; Danson, et al. (2004) Cancer Treat Rev., 30:437-49; Liu, et al. (2015) Chem. Commun., 51:9567-70; Shin, et al. (2016) Bioconjug. Chem., 27:1419-26; Zhang, et al. (2018) Org. Lett., 20:3635-8; Gonzaga, et sl. (J. Pharm. Sci. (2020) 109:3262-3281); each incorporated by reference herein). In a particular embodiment, the tumor specific or tumor up-regulated protease is cathepsin B.

In a particular embodiment, the prodrugs of the instant invention may comprise two or more different substrates for two or more different tumor specific or tumor up-regulated enzymes or proteases. For example, the prodrug may comprise orthogonal protecting groups on the protease substrate recognition sequences. The inclusion of two different substrates will require activation (e.g., simultaneously or sequentially) by two tumor enzymes or proteases. Such a prodrug further reduces the likelihood of inadvertent off-tumor activation.

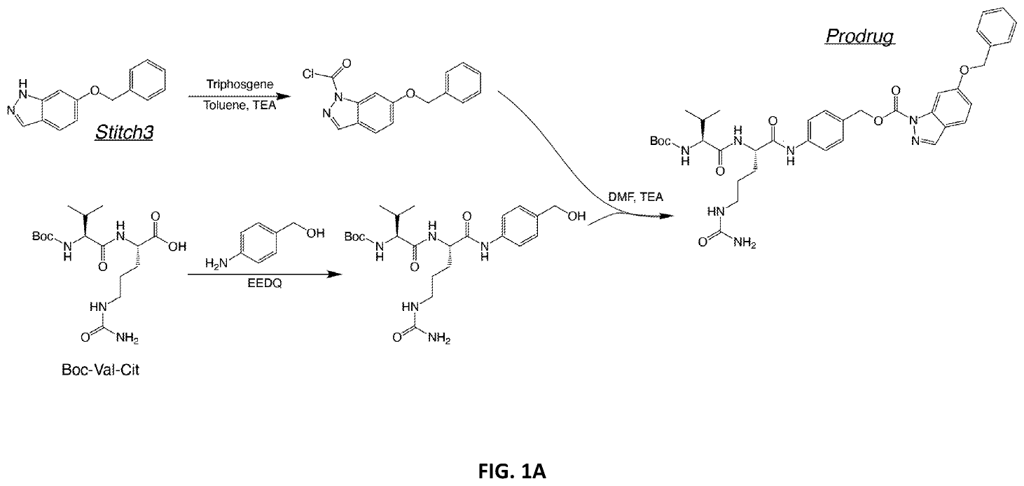

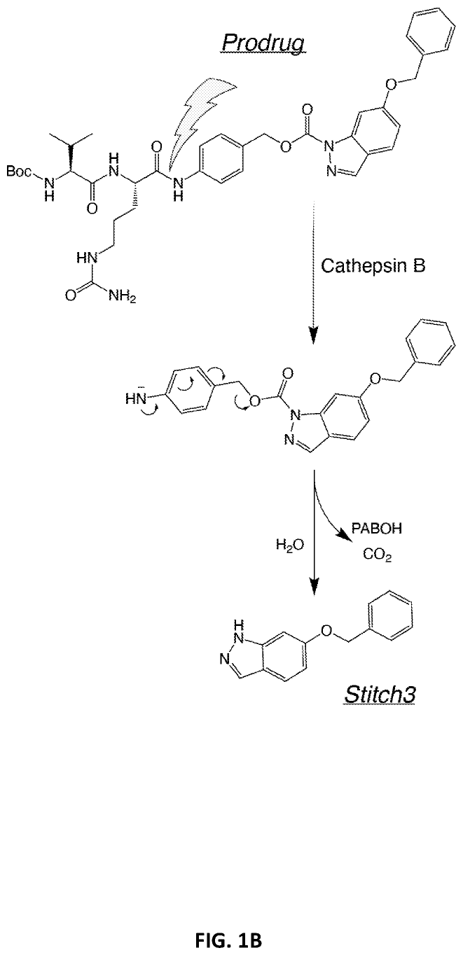

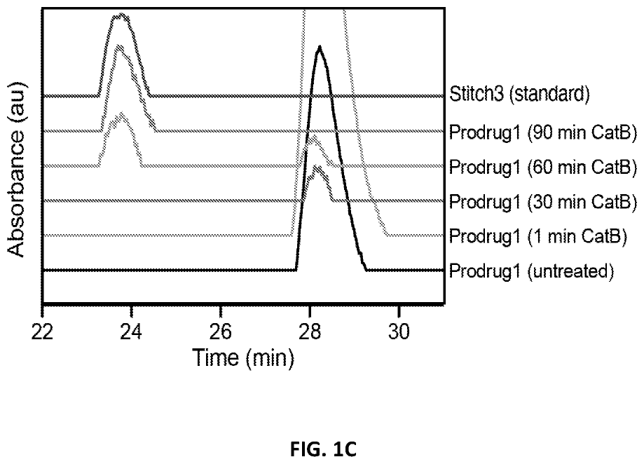

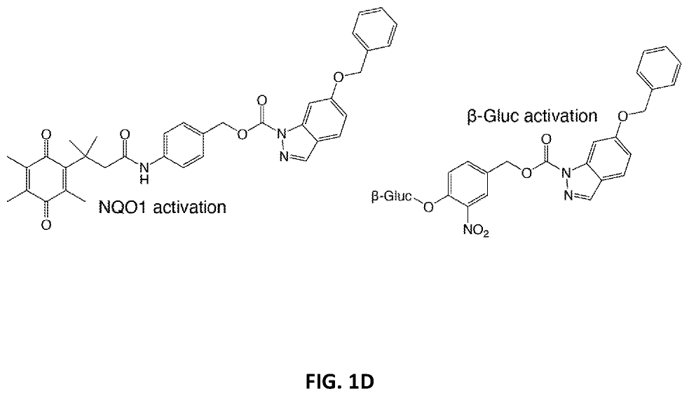

Examples of the substrates of the enzymes/proteases of the instant invention are well known in the art. In a particular embodiment, the substrate is a peptide. Peptide substrates may comprise terminal (e.g., N-terminus) modifications such as acetylation or addition of a Boc group. In a particular embodiment, the substrate for cathepsin B is the valine-citrulline dipeptide. In a particular embodiment, the substrate for legumain is D-Tyr-Tic-Ser-Asp (SEQ ID NO: 1) or D-Arg-Tic-Ser-Asp (SEQ ID NO: 2), wherein Tic is tetrahydro-isoquinoline-3-carboxylic acid. In a particular embodiment, the substrate for cathepsin L is Dap-Orn-Phe(3-Cl)-Cys(MeOBzl) (SEQ ID NO: 3), Dap-Orn-Phe(3-Cl)-Nle(OBzl) (SEQ ID NO: 4), or His-Arg-Phe-Arg (SEQ ID NO: 5; see also Poreba et al., Chem Sci. (2018) 9(8): 2113-2129). Examples of prodrugs comprising a substrate for β-glucuronidase, β-galactosidase, and DT-diaphorase (NQO1) are provided in FIG. 1D. Table 1 provides a further list of examples of enzymes/proteases and their substrates that can be used in the instant invention.

TABLE 1
List of proteases/enzymes along with examples of
their substrate (construct). Examples of thera-
peutics and disease state are also provided.

| | Construct | | | Refer- |
|---|---|---|---|---|
| Protease | (SEQ ID NO:) | Drug (X) | Disease | ences |
| uPA | TGRGPSWV (6) | | | 1 |
| uPA | D-Ala-Phe- | DOX | Cancer | 2 |
| | Lys-PABC-X | | | |
| uPA | GSGR/SAG (7) | | Cancer | 3 |
| uPA | GGSGRSANAKC | | Cancer | 4 |
| | (8) | | | |
| uPA | LGGSGRSANAI | | | 5 |
| | LE (9) | | | |
| Matrip- | LSGRSDNH | | | 6, 7 |
| tase/ | (10) | | | |
| uPA | | | | |
| Matrip- | LSGRDNH | | | 7 |
| tase/ | (11) | | | |
| uPA | | | | |
| FAPα | Z-Gly-Pro- | Dox | Cancer | 8-10 |
| | X | | | |
| | N,N- | Gemci- | | |
| | dimethyl- | tabine | | |
| | GGP-OH | | | |
| | Z-GP | | | |
| | dipeptide | | | |
| | Z-GP | Epiru- | Cancer | |
| | | bicin | | |
| Cathep- | c1F6-Val- | DOX, | Cancer | 11-17 |
| sin B | Cit-X | MMAE, | | |
| | cAC10-Val- | PTX | | |
| | Cit-X | | | |
| | Pep42-Val- | | | |
| | Cit-X | | | |
| | GAL-HPMAcp- | | | |
| | G-F-L-G-X | | | |
| | (12) | | | |
| | FRRG-X (13) | | | |
| | GFLG-X (12) | | | |
| MMP2 | GPLGV (14) | DOX | | 18-20 |
| | GPLGVRG (15) | Metho- | | |
| | PVGLIG (16) | trexate | | |
| MMP2 | Fmoc-PVGLIG | pacli- | Cancer | 21 |
| | (16) | taxel | | |
| MMP-2 | γE-P-Cit-G- | DOX | Cancer | 22, 23 |
| | Hof-Y-L-X | | | |
| | (17) | | | |
| MMP2 | GPLG/LAG | | | 24 |
| | (18) | | | |
| MMP- | GPLGVAGL | | Cancer, | 25 |
| 2/-9 | (19) | | inflam- | |
| | | | mation, | |
| | | | arterio- | |
| | | | sclerosis | |
| MMP- | GPLG/IAGQ | Dox | Cancer | 26 |
| 2/-9 | (20) | | | |
| MMP- | GPVG/LIGK | | | 27 |
| 2/-9 | (21) | | | |
| MMP-9 | γE-P-Cit-G- | DOX | Cancer | 22, 23 |
| | Hof-Y-L-X | | | |
| | (17) | | | |
| MMP-14 | γE-P-Cit-G- | DOX | Cancer | 22, 23 |
| | Hof-Y-L-X | | | |
| | (17) | | | |
| MMP-14 | PLGL (22) | | | 6 |
| MMP-14 | SLAPLGLQRR | | | 28 |
| | (23) | | | |
| MMP-14 | GRIG/ | | Cancer | 29 |
| | FLRTAKGG (24) | | | |

References:
1. WO 2010/081173;
2. De Groot, et al. (1999) J. Med. Chem., 42(25):5277-83;
3. Zuluaga, et al. (2012) Mol. Pharm., 9(6):1570-9;
4. Law, et al. (2004) Chem. Biol., 11(1):99-106;
5. Ke, et al. (1997) J. Biol. Chem., 272(33):20456-62;
6. Vasiljeva, et al. (2020) Scientific Reports 10(1):1-10;
7. Poreba, M. (2020) FEBS J., 287:1936-69;
8. Chai, et al. (2018) Acta Pharma. Sinica 39(3):415-24;
9. Sun, et al. (2019) Intl. J. Pharm., 559:48-57;
10. Wang et al. (2017) Eur. J. Pharm., 815:166-72;
11. Doronina, et al. (2003) Nat. Biotech., 21(7):778-84;
12. Jeffrey, et al. (2006) Bioorg. Med. Chem. Lett., 16(2):358-62;
13. Liang, et al. (2012) J. Controlled Rel., 160(3):618-29;
14. Sutherland, et al. (2006) J. Biol. Chem., 281(15):10540-7;
15. Francisco, et al. (2003) Blood 102(4):1458-65;
16. Shim, et al. (2019) J. Controlled Rel., 294:376-89;
17. Chen, et al. (2019) Acta biomaterialia 84:339-55;
18. Lee, et al. (2007) Eur. J. Pharm. Biopharm., 67(3):646-54;
19. Shi, et al. (2012) Intl. J. Nanomed., 7:1613;
20. Chau, et al. (2004) Bioconjugate Chem., 15(4):931-41;
21. Huang, et al. (2016) Oncotarget 7(32):52230;
22. Albright, et al. (2005) Mol. Cancer Therap., 4(5):751-60;
23. Kline, et al. (2004) Mol. Pharm., 1(1):9-22;
24. Li, et al. (2015) ACS Appl. Mater. Interfaces 7(51):28319-29;
25. Bremer, et al. (2001) Nat. Med., 7(6): 743-8;
26. Ryppa, et al. (2008) Bioconjugate Chem., 19(7):1414-22;
27. Zhang, et al. (2016) Intl. J. Nanomed., 11:1643;
28. Jabaiah, et al. (2011) Chem. Biol., 18(3):392-401;
29. Stallivieri, et al. (2018) Bioorg. Med. Chem., 26(3):688-702.

As explained hereinabove, the effector molecule of the instant invention binds the allosteric antibody and increases or restores the binding activity of the allosteric antibody. In a particular embodiment, the effector molecule is a small molecule. In a particular embodiment, the effector molecule fits or approximates the volume and electrostatics of the allosteric cavity. In a particular embodiment, the effector molecule comprises the amino acid side chains (or analog thereof) that were removed from the antibody by the mutations to make the allosteric antibody. For example, the effector molecule may comprise the removed amino acid side chains joined via a linker, particularly a short linker of about 1 to 5 atoms (e.g., a heteroatom or an alkyl optionally comprising one or more heteroatoms). Examples of effector molecule are provided in WO 2020/223273 (incorporated by reference herein; e.g., JK25 (6-phenylmethoxy-1H-indole) and those provided in FIG. 3). In a particular embodiment, the effector molecule is JK43 (Stitch3; see FIG. 1A). In a particular embodiment, the effector molecule is an analog of JK43 or a molecule provided in WO 2020/223273. For example, JK43 may be substituted (e.g., on an aromatic ring), have a carbon atom replaced with a heteroatom (e.g., N, O, or S), and/or the linker may be altered (e.g., change in length or insertion or removal of heteroatom). Examples of substituents include, for example, halo (such as F, Cl, Br, I), lower alkyl (e.g., 1-3 carbons), haloalkyl (e.g., CCl₃ or CF₃), hydroxy, methoxy, carboxyl, oxo, epoxy, amino, carbamoyl (e.g., NH₂C(═O)—), urea (—NHCONH₂), ether, ester, thioester, nitrile, nitro, amide, carbonyl, carboxylate and thiol. In certain embodiments, the substrate (e.g., via a linker) is attached to an available nitrogen of JK43 (e.g., as depicted in FIG. 1) in the prodrug.

Compositions comprising a prodrug are also encompassed by the instant invention. In a particular embodiment, the composition comprises at least one prodrug of the instant invention and at least one pharmaceutically acceptable carrier. In a particular embodiment, the composition comprises nanoparticles containing at least one prodrug of the instant invention and at least one pharmaceutically acceptable carrier.

Nanoparticles comprising the prodrugs of the instant invention are also encompassed. Nanoparticles have been effectively used to deliver chemotherapeutics to the tumor microenvironment (Luo, et al. (2014) Trends Pharmacol. Sci., 35:556-66; Lin, et al. (2019) Future Med. Chem., 11:2131-50; Dheer, et al. (2019) Adv. Drug Deliv. Rev., 151-152:130-51). Use of a nanocarrier/nanoparticle format may also yield ancillary benefits with respect to enhanced stability, solubility, and/or bioavailability.

Allosteric antibodies which can be bound by the effector molecules of the instant invention to increase or restore the binding activity of the allosteric antibody generally comprises at least two amino acid mutations (e.g., substitutions). Examples of allosteric antibodies are described in WO 2020/223273 (incorporated by reference herein). In a particular embodiment, the allosteric antibody cannot bind or has diminished binding affinity (e.g., at least about 10 fold lower, at least about 50 fold lower, at least about 100 fold lower or more) for its antigen than the wild-type/unmutated antibody. The binding affinity of the allosteric antibody is increased (e.g., at least about 2 fold, at least about 5 fold, at least about 10 fold, at least about 20 fold, at least about 50 fold, at least about 100 fold or more) or restored (e.g., to near or at wild-type levels) by binding of an effector molecule. The use of the effector molecule allows for spatial and/or temporal control of the activity (e.g., antigen binding) of the antibody.

The allosteric antibodies of the instant invention may have one, two, three, four, five or, particularly, all six of the following characteristics. First, the amino acid mutations form a space or cavity within the allosteric antibody (i.e., the mutated sidechains must be in close proximity (in three-dimensional space) to one other, so that the resulting cavity will be contiguous). Second, the amino acid mutations result in the removal of at least 12 heavy atoms (e.g., non-hydrogen atoms) from the antibody. Third, at least one of the mutated amino acids comprises an aromatic side chain (e.g., at least of the mutated/substituted amino acids is phenylalanine, tyrosine, or tryptophan). Fourth, the mutated residues are located at an interface between two domains or chains of the antibody (e.g., between the two domains of an scFv or between the heavy chain and light chain of an antibody). Fifth, the amino acid mutations are not within the complementarity determining regions (CDRs) of the antibody. Sixth, the amino acid mutations are within the variable regions (e.g., within the framework regions of the variable regions).

In a particular embodiment, the allosteric antibody comprises the amino acid substitutions of at least two amino acids with glycine and/or alanine, particularly glycine. In a particular embodiment, the allosteric antibody comprises a Trp110Gly substitution in the heavy chain and a Phe98Gly substitution in its light chain (e.g., based on amino acid positioning in the 4D5Flu antibody). In a particular embodiment, the allosteric antibody comprises a Trp110Gly substitution in the heavy chain and a Tyr36Gly substitution in its light chain (e.g., based on amino acid positioning in the 4D5Flu antibody). In a particular embodiment, the allosteric antibody comprises a Trp110Gly substitution and a Val37Ala substitution in the heavy chain and a Phe98Gly substitution in its light chain (e.g., based on amino acid positioning in the 4D5Flu antibody). In a particular embodiment, the allosteric antibody comprises a Trp110Gly substitution and a Tyr95Ala substitution in the heavy chain and a Phe98Ala substitution in its light chain (e.g., based on amino acid positioning in the 4D5Flu antibody). In a particular embodiment, the allosteric antibody comprises a Trp47Ala substitution and a Val37Ala substitution in the heavy chain and a Phe98Gly substitution in its light chain (e.g., based on amino acid positioning in the 4D5Flu antibody). The instant invention also encompasses allosteric antibodies wherein any or all glycine substitution is replaced with an alanine substitution and/or any or all alanine substitutions are replaced with a glycine substitution. For example, the instant invention encompasses an allosteric antibody wherein Trp110 and Val37 in the heavy chain and Phe98 in its light chain (e.g., based on amino acid positioning in the 4D5Flu antibody) are each independently substituted with either an alanine or a glycine. As noted, the above amino acid positions are based on the positioning in the 4D5Flu antibody (sequential numbering). With regard to IMGT numbering, the amino acids are V_{L}F118, V_{H}V42, and V_{H}W118. With regard to Chothia numbering, the amino acids are V_{L}F98, V_{H}V37, and V_{H}W103. Accordingly, the instant invention also encompasses allosteric antibodies wherein the amino acid positions are defined by these numbering systems (e.g., IMGT: V_{L}F118G, V_{H}V42A, and V_{H}W118G; Chothia: V_{L}F98G, V_{H}V37A, and V_{H}W103G).

The amino acid numbering (provided above in the 4D5Flu antibody) may vary in different antibodies. As such, the corresponding amino acid may be substituted (e.g., the same amino acid but at a different numbered position having the same general three-dimensional location within the antibody). The skilled artisan can determine the location of the amino acids to be substituted through an alignment of the amino acid sequences and/or three-dimensional modeling of the amino acids locations within the antibody. In a particular embodiment, the V_{L}F98 or corresponding amino acid is contained within the sequence PX₁TFGX₂G (SEQ ID NO: 25; particularly wherein X₁ is W, R, Y, or A and/or X₂ is G or Q). In a particular embodiment, the V_{H}V37 or corresponding amino acid is contained within the sequence X₁X₂WVX₃QX₄ (SEQ ID NO: 26; particularly wherein X₁ is M or I; X₂ is H or N; X₃ is R or K; and/or X₄ is S or A). In a particular embodiment, the V_{H}W110 or corresponding amino acid is contained within the sequence X₁DX₂WGX₃G (SEQ ID NO: 27; particularly wherein X₁ is M, F, L, or D; X₂ is Y or V; and/or X₃ is Q or A).

The allosteric antibodies can be a full-length antibody (e.g., IgG antibody) or a fragment thereof, particularly an antigen binding fragment thereof. The allosteric antibody may be a monoclonal antibody. The antibody may be a naturally occurring antibody or may be a synthetic or modified antibody (e.g., a recombinantly generated antibody; a chimeric antibody; a bispecific antibody; a humanized antibody; a camelid antibody; and the like). The antibody may comprise at least one purification tag. In a particular embodiment, the allosteric antibody is an antibody fragment. Antibody fragments include, without limitation, immunoglobulin fragments including, without limitation: Fab, Fab′, F(ab′)2, and F(v); and fusions (e.g., via a linker) of these immunoglobulin fragments including, without limitation: scFv, scFv₂, scFv-Fc, minibody, diabody, triabody, and tetrabody. The antibody may also be a protein (e.g., a fusion protein) comprising at least one antibody or antibody fragment. In a particular embodiment, the allosteric antibody is or comprises a Fab fragment. In a particular embodiment, the allosteric antibody is or comprises an scFv.

The antibody molecules of the invention may be prepared using a variety of methods known in the art. Polyclonal and monoclonal antibodies may be prepared, for example, as described in Current Protocols in Molecular Biology, Ausubel et al. eds. Antibodies may be prepared by chemical cross-linking, hybrid hybridoma techniques and by expression of recombinant antibody fragments expressed in host cells, such as bacteria or yeast cells. In one embodiment of the invention, the antibody molecules are produced by expression of recombinant antibody or antibody fragments in host cells. The nucleic acid molecules encoding the antibody may be inserted into expression vectors and introduced into host cells. The resulting antibody molecules are then isolated and purified from the expression system. The antibodies optionally comprise a purification tag by which the antibody can be purified.

The purity of the antibody molecules of the invention may be assessed using standard methods known to those of skill in the art, including, but not limited to, ELISA, immunohistochemistry, ion-exchange chromatography, affinity chromatography, immobilized metal affinity chromatography (IMAC), size exclusion chromatography, polyacrylamide gel electrophoresis (PAGE), western blotting, surface plasmon resonance and mass spectroscopy.

The allosteric antibodies can bind any antigen. For example, the allosteric antibody may be, without limitation, an anti-TNF-alpha antibody, an anti-VEGF-A antibody, or an antibody against an immune checkpoint (e.g., a checkpoint inhibitor). Immune checkpoints include, without limitation, cytotoxic T-lymphocyte-associated protein 4 (CTLA-4), programmed cell death protein 1 (PD-1), and programmed death-ligand 1 (PD-L1). In a particular embodiment, the allosteric antibody is based on (e.g., a mutation of) a therapeutic antibody. Examples of therapeutic antibodies are set forth below.

In a particular embodiment, the antibody to be mutated/substituted comprises amino acids V_{L}F98, V_{H}V37, and V_{H}W110 (based on amino acid positioning in the 4D5Flu antibody) or the same amino acids at corresponding positions within the antibody. In a particular embodiment, the resulting allosteric antibody comprises the triple mutation V_{L}F98G/V_{H}V37A/V_{H}W110G or the corresponding amino acid substitutions of the same amino acids at the corresponding position in the antibody. Amino acids V_{L}F98, V_{H}V37, and V_{H}W110 (e.g., the constellation thereof in approximately the same positions as the 4D5Flu antibody) can be found in most antibodies. For example, therapeutic antibodies which include amino acids corresponding to V_{L}F98, V_{H}V37, and V_{H}W110 include, without limitation: abagovomab, abelacimab, abituzumab, abrilumab, actoxumab, adalimumab, aducanumab, afasevikumab, afutuzumab, alacizumab, alemtuzumab, alirocumab, amatuximab, amivantamab, andecaliximab, anetumab, anifrolumab, anrukinzumab, apamistamab, aprutumab, astegolimab, atezolizumab, atinumab, atoltivimab, avdoralimab, avelumab, avizakimab, azintuxizumab, balstilimab, bapineuzumab, bavituximab, bedinvetmab, befovacimab, begelomab, belantamab, belimumab, bemarituzumab, benralizumab, benufutamab, bermekimab, bersanlimab, bevacizumab, bezlotoxumab, bifikafusp, bimagrumab, bimekizumab, bintrafusp, blinatumomab, blontuvetmab, blosozumab, bococizumab, brazikumab, brentuximab, briakinumab, brodalumab, brolucizumab, brontictuzumab, budigalimab, burosumab, cabiralizumab, camidanlumab, camrelizumab, canakinumab, cantuzumab, carlumab, carotuximab, cemiplimab, cergutuzumab, certolizumab, cetrelimab, cetuximab, cibisatamab, cinpanemab, citatuzumab, cixutumumab, claudiximab, clazakizumab, clivatuzumab, cobolimab, codrituzumab, cofetuzumab, coltuximab, concizumab, cosibelimab, crenezumab, crizanlizumab, crotedumab, crovalimab, cusatuzumab, dacetuzumab, daclizumab, dapirolizumab, daratumumab, dectrekumab, demcizumab, denosumab, dezamizumab, dilpacimab, dinutuximab, diridavumab, disitamab, domagrozumab, donanemab, dostarlimab, drozitumab, duligotuzumab, dupilumab, durvalumab, dusigitumab, eculizumab, edrecolomab, efalizumab, efungumab, eldelumab, elezanumab, elgemtumab, elotuzumab, emactuzumab, emapalumab, emicizumab, emibetuzumab, enapotamab, enavatuzumab, encelimab, enfortumab, enoblituzumab, enokizumab, enoticumab, ensituximab, epcoritamab, epratuzumab, eptinezumab, erenumab, etaracizumab, etokimab, etrolizumab, evinacumab, evolocumab, faricimab, farletuzumab, fasinumab, fezakinumab, fianlimab, fibatuzumab, ficlatuzumab, figitumumab, firivumab, flanvotumab, fletikumab, flotetuzumab, fontolizumab, foralumab, foravirumab, fremanezumab, fresolimumab, frovocimab, frunevetmab, fulranumab, futuximab, galcanezumab, gancotamab, ganitumab, gantenerumab, garadacimab, gatipotuzumab, gatralimab, gedivumab, gemtuzumab, gilvetmab, girentuximab, glenzocimab, golimumab, gomiliximab, gosuranemab, gremubamab, guselkumab, ibalizumab, icrucumab, ieramilimab, ifabotuzumab, iladatuzumab, imalumab, imaprelimab, imgatuzumab, inclacumab, indatuximab, inebilizumab, infliximab, inotuzumab, intetumumab, ipilimumab, isatuximab, iscalimab, istiratumab, ivuxolimab, ixekizumab, labetuzumab, lacutamab, ladiratuzumab, lambrolizumab, lampalizumab, lanadelumab, landogrozumab, laprituximab, larcaviximab, lenvervimab, lenzilumab, leronlimab, lesofavumab, levilimab, lexatumumab, lifastuzumab, ligelizumab, lilotomab, lintuzumab, lirilumab, lodapolimab, lodelcizumab, lokivetmab, loncastuximab, lorukafusp, lorvotuzumab, lucatumumab, lumiliximab, lumretuzumab, lupartumab, lutikizumab, maftivimab, magrolimab, manelimab, margetuximab, marstacimab, matuzumab, mavezelimab, mavrilimumab, mepolizumab, mezagitamab, mirikizumab, mirvetuximab, mitazalimab, modotuximab, mogamulizumab, monalizumab, mosunetuzumab, moxetumomab, murlentamab, muromonab, namilumab, naptumomab, naratuximab, narnatumab, natalizumab, navivumab, navicixizumab, naxitamab, nemolizumab, nesvacumab, nidanilimab, nimacimab, nimotuzumab, nirsevimab, nivolumab, nurulimab, obexelimab, obiltoxaximab, obinutuzumab, ocaratuzumab, ocrelizumab, odesivimab, odronextamab, ofatumumab, oleclumab, olendalizumab, olinvacimab, olokizumab, omburtamab, onartuzumab, onfekafusp, ontamalimab, ontuxizumab, onvatilimab, opicinumab, oportuzumab, orilanolimab, orticumab, osocimab, otelixizumab, otilimab, otlertuzumab, oxelumab, ozanezumab, pabinafusp, pacmilimab, pamrevlumab, pankomab, panobacumab, parsatuzumab, pasotuxizumab, pateclizumab, pembrolizumab, pepinemab, perakizumab, pertuzumab, petosemtamab, pidilizumab, pinatuzumab, plamotamab, plozalizumab, pogalizumab, polatuzumab, ponezumab, porgaviximab, prasinezumab, prezalumab, pritoxaximab, prolgolimab, quetmolimab, quilizumab, racotumomab, radretumab, rafivirumab, ralpancizumab, ramucirumab, ranevetmab, ranibizumab, ravagalimab, ravulizumab, refanezumab, relfovetmab, remtolumab, retifanlimab, rituximab, rivabazumab, robatumumab, roledumab, rolinsatamab, romilkimab, romosozumab, rontalizumab, rosmantuzumab, rovalpituzumab, rozanolixizumab, rozipafusp, ruplizumab, sacituzumab, samalizumab, samrotamab, sapelizumab, sarilumab, sasanlimab, satralizumab, secukinumab, selicrelumab, semorinemab, seribantumab, setoxaximab, setrusumab, sifalimumab, simlukafusp, simtuzumab, sintilimab, sirtratumab, sirukumab, sofituzumab, solanezumab, solitomab, spartalizumab, spesolimab, suptavumab, sutimlimab, suvratoxumab, tabituximab, tadocizumab, tafasitamab, talquetamab, tamrintamab, tanibirumab, tarextumab, tebentafusp, telisotuzumab, temelimab, tenatumomab, teplizumab, tepoditamab, teprotumumab, tesidolumab, tezepelumab, ticilimumab, tigatuzumab, tilavonemab, tildrakizumab, tilvestamab, timigutuzumab, timolumab, tisotumab, tocilizumab, tomaralimab, tomuzotuximab, toripalimab, tosatoxumab, tralokinumab, trastuzumab, tremelimumab, trevogrumab, ublituximab, ulocuplumab, ustekinumab, utomilumab, vadastuximab, valanafusp, vanalimab, vandortuzumab, vantictumab, vanucizumab, varisacumab, varlilumab, vedolizumab, veltuzumab, vesencumab, vibecotamab, vibostolimab, visilizumab, vofatamab, volagidemab, vonlerolizumab, vopratelimab, vorsetuzumab, vunakizumab, xentuzumab, zagotenemab, zalifrelimab, zalutumumab, zampilimab, zanidatamab, zatuximab, zelminemab, zenocutuzumab, ziltivekimab, zolbetuximab, and zolimomab. In a particular embodiment, the therapeutic antibody is selected from the group consisting of: muromonab, abciximab, capromab, nofetumomab, daclizumab, rituximab, basiliximab, etanercept, infliximab, palivizumab, trastuzumab, arcitumomab, alemtuzumab, adalimumab, ibritumomab, alefacept, tositumomab, bevacizumab, cetuximab, omalizumab, abatacept, natalizumab, panitumumab, ranibizumab, eculizumab, certolizumab, rilonacept, ustekinumab, canakinumab, golimumab, ofatumumab, denosumab, tocilizumab, aflibercept, brentuximab, belatacept, belimumab, ipilimumab, pertuzumab, raxibacumab, obinutuzumab/afutuzumab, adotrastuzumab, blinatumomab, nivolumab, pembrolizumab/lambrolizumab, ramucirumab, siltuximab, vedolizumab, alirocumab, daratumumab, dinutuximab, elotuzumab, evolocumab, idarucizumab, mepolizumab, necitumumab, secukinumab, atezolizumab, infliximab, ixekizumab, obiltoxaximab, and reslizumab. In a particular embodiment, the therapeutic antibody is adalimumab. In a particular embodiment, the therapeutic antibody is ipilimumab.

The allosteric antibodies may be further modified. For example, the allosteric antibodies may be humanized. In a particular embodiment, the antibodies (or a portion thereof) are inserted into the backbone of an antibody or antibody fragment construct. For example, the variable light domain and/or variable heavy domain of the antibodies of the instant invention may be inserted into another antibody construct. Methods for recombinantly producing antibodies are well-known in the art. Indeed, commercial vectors for certain antibody and antibody fragment constructs are available.

The allosteric antibodies may also be conjugated/linked to other components. For example, the antibodies may be operably linked (e.g., covalently linked, optionally, through a linker) to at least one detectable agent, imaging agent, contrast agent, immunosuppressant, or anti-inflammatory agent. The antibodies of the instant invention may also comprise at least one purification tag (e.g., a His-tag).

In accordance with another aspect of the instant invention, methods of modulating antibody activity are provided. The methods can be performed in vitro or in vivo. In a particular embodiment, the methods are used to inhibit (e.g., reduce or slow), treat, and/or prevent a disease or disorder (e.g., cancer) in a subject are provided. In a particular embodiment, the methods reduce the size of a tumor in a subject.

Generally, the methods of the instant invention comprise administering to a subject or cell an allosteric antibody and prodrug. In a particular embodiment, the allosteric antibody and prodrug are contained in separate compositions comprising a pharmaceutically acceptable carrier. In a particular embodiment, the allosteric antibody and the prodrug are administered at different times. In a particular embodiment, the allosteric antibody and the prodrug are administered to different locations and/or by different means, particularly within a subject.

In a particular embodiment, the allosteric antibody and prodrug are paired to treat a particular cancer. For example, if the allosteric antibody is a variant of ipilimumab and used to treat melanoma, the prodrug should have a protecting group recognized by an enzyme or protease that is specific to melanoma, such as cathepsin B.

With regard to in vivo or therapeutic methods, the allosteric antibody (e.g., based on a therapeutic antibody) can be administered to the subject by any means. In a particular embodiment, the allosteric antibody is administered systemically. In a particular embodiment, the allosteric antibody is administered intravenously, intramuscularly, or subcutaneously. The prodrug may be administered simultaneously and/or at different times (e.g., consecutively) than the allosteric antibody. In a particular embodiment, the prodrug is administered at a different time and/or different location than the allosteric antibody. For example, in the context of treating cancer, the allosteric antibody may be administered systemically and the prodrug may be administered systemically or locally (e.g., by direct injection to the or near the cancer or tumor), optionally at a later timepoint.

Except insofar as any conventional carrier is incompatible with the compound to be administered, its use in the pharmaceutical composition is contemplated. In a particular embodiment, the carrier is a pharmaceutically acceptable carrier for transdermal, intravenous, intramuscular, or subcutaneous administration. The instant invention also encompasses kits comprising a composition comprising an allosteric antibody and at least one carrier (e.g., a pharmaceutically acceptable carrier) and/or a composition comprising a prodrug and at least one carrier (e.g., a pharmaceutically acceptable carrier).

As explained hereinabove, the compositions of the instant invention are useful for treating a disease or disorder (e.g., cancer). A therapeutically effective amount of the composition may be administered to a subject in need thereof. The dosages, methods, and times of administration are readily determinable by persons skilled in the art, given the teachings provided herein.

The components as described herein will generally be administered to a patient as a pharmaceutical preparation. The term “patient” or “subject” as used herein refers to human or animal subjects. The components of the instant invention may be employed therapeutically, under the guidance of a physician for the treatment of the indicated disease or disorder.

The pharmaceutical preparation comprising the components of the invention may be conveniently formulated for administration with an acceptable medium (e.g., pharmaceutically acceptable carrier) such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the agents in the chosen medium may be varied and the medium may be chosen based on the desired route of administration of the pharmaceutical preparation. Except insofar as any conventional media or agent is incompatible with the agents to be administered, its use in the pharmaceutical preparation is contemplated.

The compositions of the present invention can be administered by any suitable route, for example, by injection (e.g., for local (direct) or systemic administration), oral, pulmonary, topical, nasal or other modes of administration. The composition may be administered by any suitable means, including parenteral, intramuscular, intravenous, intraarterial, intraperitoneal, subcutaneous, topical, inhalatory, transdermal, intrapulmonary, intraareterial, intrarectal, intramuscular, and intranasal administration. In a particular embodiment, the composition is administered directly to the blood stream (e.g., intravenously). In a particular embodiment, the composition is administered by direct injection. In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., polysorbate 80), antioxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington: The Science and Practice of Pharmacy, 21st edition, Philadelphia, PA. Lippincott Williams & Wilkins. The pharmaceutical composition of the present invention can be prepared, for example, in liquid form, or can be in dried powder form (e.g., lyophilized for later reconstitution).

As used herein, “pharmaceutically acceptable carrier” includes any and all solvents, dispersion media and the like which may be appropriate for the desired route of administration of the pharmaceutical preparation, as exemplified in the preceding paragraph. The use of such media for pharmaceutically active substances is known in the art. Except insofar as any conventional media or agent is incompatible with the molecules to be administered, its use in the pharmaceutical preparation is contemplated.

Pharmaceutical compositions containing a compound of the present invention as the active ingredient in intimate admixture with a pharmaceutical carrier can be prepared according to conventional pharmaceutical compounding techniques. The carrier may take a wide variety of forms depending on the form of preparation desired for administration, e.g., intravenous. Injectable suspensions may be prepared, in which case appropriate liquid carriers, suspending agents and the like may be employed. Pharmaceutical preparations for injection are known in the art. If injection is selected as a method for administering the therapy, steps should be taken to ensure that sufficient amounts of the molecules reach their target cells to exert a biological effect.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art. The appropriate dosage unit for the administration of the molecules of the instant invention may be determined by evaluating the toxicity of the molecules in animal models. Various concentrations of pharmaceutical preparations may be administered to mice with transplanted human tumors, and the minimal and maximal dosages may be determined based on the results of significant reduction of tumor size and side effects as a result of the treatment. Appropriate dosage unit may also be determined by assessing the efficacy of the treatment in combination with other standard therapies.

The pharmaceutical preparation comprising the molecules of the instant invention may be administered at appropriate intervals, for example, at least twice a day or more until the pathological symptoms are reduced or alleviated, after which the dosage may be reduced to a maintenance level. The appropriate interval in a particular case would normally depend on the condition of the patient.

### Definitions

The singular forms “a,” “an,” and “the” include plural referents unless the context clearly dictates otherwise.

The terms “isolated” is not meant to exclude artificial or synthetic mixtures with other compounds or materials, or the presence of impurities that do not interfere with the fundamental activity, and that may be present, for example, due to incomplete purification, or the addition of stabilizers.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxilliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in Remington: The Science and Practice of Pharmacy, (Lippincott, Williams and Wilkins); Liberman, et al., Eds., Pharmaceutical Dosage Forms, Marcel Decker, New York, N.Y.; and Rowe, et al., Eds., Handbook of Pharmaceutical Excipients, Pharmaceutical Pr.

The term “treat” as used herein refers to any type of treatment that imparts a benefit to a patient suffering from an injury, including improvement in the condition of the patient (e.g., in one or more symptoms), delay in the progression of the condition, etc.

As used herein, the term “prevent” refers to the prophylactic treatment of a subject who is at risk of developing a condition and/or sustaining an injury, resulting in a decrease in the probability that the subject will develop conditions associated with a disease or disorder (e.g., cancer).

A “therapeutically effective amount” of a compound or a pharmaceutical composition refers to an amount effective to prevent, inhibit, or treat a particular injury and/or the symptoms thereof. For example, “therapeutically effective amount” may refer to an amount sufficient to modulate the pathology associated with a disease or disorder (e.g., cancer).

As used herein, the term “subject” refers to an animal, particularly a mammal, particularly a human.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, amino acids, or nucleic acids.

An “antibody” or “antibody molecule” is any immunoglobulin, including antibodies and fragments thereof, that binds to a specific antigen. As used herein, antibody or antibody molecule contemplates intact immunoglobulin molecules, immunologically active portions/fragment (e.g., antigen binding portion/fragment) of an immunoglobulin molecule, and fusions of immunologically active portions of an immunoglobulin molecule. Antibody fragments (e.g., antigen binding antibody fragments) include, without limitation, immunoglobulin fragments including, without limitation: single domain (Dab; e.g., single variable light or heavy chain domain), Fab, Fab′, F(ab′)₂, and F(v); and fusions (e.g., via a linker) of these immunoglobulin fragments including, without limitation: scFv, scFv₂, scFv-Fc, minibody, diabody, triabody, and tetrabody.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

As used herein, “prodrug” means any compound that when administered to a biological system generates the drug substance, i.e., active ingredient, as a result of spontaneous chemical reaction(s), enzyme catalyzed chemical reaction(s), photolysis, and/or metabolic chemical reaction(s). A prodrug is, thus, a covalently modified analog or latent form of a compound, typically with therapeutic activity.

“Linker” refers to a chemical moiety comprising a chain of atoms that covalently attach at least two compounds. The linker can be linked to any synthetically feasible position of the compounds, but preferably in such a manner as to avoid blocking the compounds desired activity. Linkers are generally known in the art. In a particular embodiment, the linker may contain from 1 to about 100 atoms, 1 to about 50 atoms, 1 to about 25 atoms, or from 1 to about 10 atoms.

The term “kit” generally refers to an assembly of materials and/or reagents that is used for a particular application(s). The materials and/or reagents can be provided in the same or in separate containers, and in liquid or in lyophilized form. The amounts and proportions of materials and/or reagents provided in the kit can be selected so as to provide optimum results for a particular application(s).

A “conservative amino acid substitution” refers to amino acid substitutions that substitute functionally-equivalent or similar amino acids. For example, an amino acid may be substituted with an amino acid having a similar polarity, charge, size, and/or aromaticity. In a particular embodiment, a conservative substitutions is a substitution within the same group of amino acids such as non-polar amino acids (e.g., Trp, Phe, Met, Leu, Ile, Val, Ala, Pro, Gly), uncharged polar amino acids (e.g., Ser, Thr, Asn, Gln, Tyr, Cys), acidic amino acids (e.g., Asp, Glu), basic amino acids (e.g., Arg, Lys, His), beta-branched amino acids (e.g., Thr, Val, Ile), and aromatic amino acids (e.g., Trp, Tyr, Phe).

The following example is provided to illustrate various embodiments of the present invention. The example is illustrative and not intended to limit the invention in any way.

### EXAMPLE

A prodrug which is activated by cathepsin B was synthesized. Cathepsin B is involved in various pathologies and oncogenic processes and cathepsin B overexpression is correlated with invasive and metastatic phenotypes in cancers (Gondi et al., Expert Opin. Ther. Targets (2013) 17(3):281-291). FIG. 1A provides a schematic for the synthesis of a prodrug comprising the effector molecule Stitch3, a self-immolating linker comprising a dipeptide (valine-citrulline) recognized by cathepsin B, and a protecting group (tert-butyloxycarbonyl (Boc)). The Boc protecting group is optional and not required for activity. Briefly, Stitch3 was reacted with triphosgene (Cl₃COCOOCCl₃) in the presence of toluene and triethylamine (TEA). Separately, the Boc-protected dipeptide (Val-Cit) was reacted with 1-amino-4-(hydroxymethyl)benzene in N-ethoxycarbonyl-2-ethoxy-1,2-dihydroquinoline (EEDQ). The two resultant products were combined in dimethylformamide and TEA to yield the desired prodrug. The resulting compound was stable in aqueous buffer. While FIG. 1A depicts the addition of the linker to a nitrogen of the indazole group of Stitch3, the linker may be attached to any chemically feasible position of Stitch3 (e.g., the other nitrogen of the indazole group of Stitch3).

FIG. 1B provides a schematic of the activation of the prodrug. Upon treatment with cathepsin B to cleave the dipeptide, a cascading re-organization leads to spontaneous liberation of the p-aminobenzylcarbonyl (PABC) linker. The conversion of the prodrug to Stitch 3 by cathepsin B was confirmed by mass spectrometry. Briefly, the prodrug was treated with 40 nm cathepsin B (Dubowchik, et al., Bioorg. Med. Chem. Lett. (1998) 8:3341-6; Karnthaler-Benbakka, et al., Chem. Biodivers. (2019) 16:e1800520). As seen in FIG. 1C, mass spectrometry confirmed the expected m/z change from 730 Da (prodrug) to 225 Da (Stitch3). The successful conversion back to Stitch3 was observed with a half-life faster than 60 minutes.

In addition to the above, an in vitro CTLA-4/CD80 HTRF (homogeneous time resolved fluorescence) binding assay was performed with cathepsin B activatable prodrug. Specifically, the HTRF assay was used to assess the ability of antibodies to block the CTLA-4/CD80 interaction using a CTLA-4/CD80 binding assay kit (cat. no. 64CTLA80PEG; Cisbio (Codolet, France)). Briefly, 2 μL of standard or wild-type antibody (5GSWT—an anti-CTLA-4 scFv construct based on ipilimumab with a GG(GGSGG)₅GG linker (SEQ ID NO: 30)) and a triple-mutant antibody (5GS 3′M (Rip3)—the 5GSWT scFv with the triple mutation (V_{L}F98G/V_{H}V37A/V_{H}W110G)) with a final concentration ranging from (412.5, 206, 103.1, 51.5, 25.1, 12.8, 6.4 and 3.2 nM) were used. The amino acid sequences of the light chain variable domain and the heavy chain variable domain of ipilimumab are (amino acids which correspond to VLF98, VHV37, and VHW110 in 4D5Flu are underlined): V_{L} from ipilimumab:

(SEQ ID NO: 28)
EIVLTQSPGTLSLSPGERATLSCRASQSVGSSYLAWYQQKPGQAPRLLIY
GAFSRATGIPDRFSGSGSGTDFTLTISRLEPEDFAVYYCQQYGSSPWTFG
QGTKVEIKR

V_{H} from Ipilimumab:

(SEQ ID NO: 29)
QVQLVESGGGVVQPGRSLRLSCAASGFTFSSYTMHWVRQAPGKGLEWVTF
ISYDGNNKYYADSVKGRFTISRDNSKNTLYLQMNSLRAEDTAIYYCARTG
WLGPFDYWGQGTLVTVSS.

To the above, 4 μL of Tag1-CD80 protein and 4 μL of Tag2-CTLA-4 were added into a black 384-well low volume plate and then incubated at 37° C. for 10 minutes. Up to 75 μM JK43 and prodrug was used in the assay. Cathepsin B (human liver, 0.47 mg/ml, 324 u/mg; Merck) was used with the final concentration of 2.29×10⁻⁵ mg/ml, which cleaves the prodrug and releases functional ligand JK43 in the assay buffer. To make the working stock of cathepsin B, 1 μl from stock was diluted in 9 μl of PBS (total of 10 μl volume). Then 2 μl of activation buffer (30 mM DTT/15 mM EDTA-Na₂ in H₂O) was added to 300 μl of PPI Europium detection buffer (CTLA-4/CD80 binding assay kit). 0.6 μl activated cathepsin B was added to each sample. Following that, 5 μL of anti-Tag1-Europium Cryptate (HTRF donor), 5 μL of anti-Tag2-d2 reagent (HTRF acceptor) were premixed and added for each sample in the 384-well plate together. These were used to measure the fluorescence energy transfer between donor Anti-Tag1 Eu Cryptate and acceptor Anti-Tag d2 reagent. Data was collected at different time points (1, 3, 8, 12 and 24 hours) and the FRET signals were measured on a envision multimode plate reader (PerkinElmer, Shelton, CT) using 340 nm as the excitation wavelength, a 620 nm filter for the Eu donor fluorescence detection, and a 665 nm filter for the acceptor fluorescence detection. HTRF signals were calculated as a ratio as follows: (intensity of 665 nm)/(intensity of 620 nm)×10,000, which was plotted against the WT/triple mutant antibody (5GSWT and 5GS 3′M (Rip3)) concentration to derive the EC50.

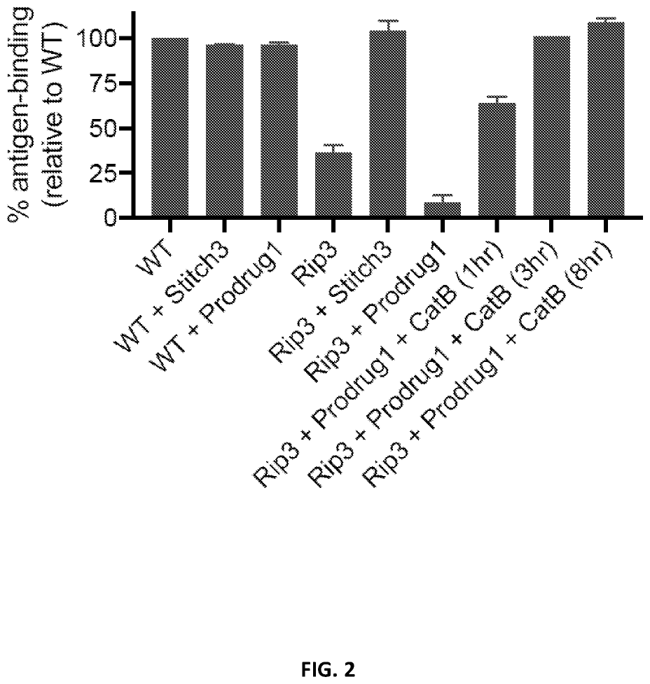

As seen in FIG. 2, the addition of Stitch3 rescues the activity of the triple mutant scFv variant (Rip3). However, the addition of the prodrug (Prodrug1) does not rescue the activity of the triple mutant scFv variant (Rip3). Surprisingly, the addition of the prodrug actually reduces the basal activity of the triple mutant scFv variant (Rip3). Without being bound by theory, the prodrug may occupy part or half of the designed binding site, thereby preventing the triple mutant scFv from transiently visiting the active conformation (as it does in the unbound form). Significantly, the addition of cathepsin B to the assay converts prodrug1 to Stitch3, which rescues the binding activity of the triple mutant scFv variant, thereby rescuing CTLA-4 inhibition.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A prodrug comprising an effector molecule and a protecting group,
wherein said effector molecule binds an allosteric antibody and increases or restores its binding activity,
wherein said protecting group comprises a substrate for a cleaving enzyme and a self-immolating linker, and
wherein said substrate for a cleaving enzyme is covalently attached to said effector molecule by said self-immolating linker.

2. The prodrug of claim 1, wherein said cleaving enzyme is tumor specific or upregulated in the tumor microenvironment.

3. The prodrug of claim 1, wherein said cleaving enzyme is cathepsin B.

4. The prodrug of claim 1, wherein said substrate comprises valine-citrulline.

5. The prodrug of claim 1, wherein said effector molecule comprises 6-phenylmethoxy-1H-indazole.

6. The prodrug of claim 1, wherein said self-immolating linker comprises p-aminobenzyl.

7. The prodrug of claim 1, wherein said effector molecule comprises 6-phenylmethoxy-1H-indazole and said self-immolating linker comprises p-aminobenzyl.

8. The prodrug of claim 7, wherein said substrate comprises valine-citrulline.

9. A method of modulating the activity of an allosteric antibody, said method comprising contacting the allosteric antibody with the prodrug of any one of claims 1-8.

10. The method of claim 9, wherein said allosteric antibody and effector molecule are administered to a subject.

11. The method of claim 9, wherein said allosteric antibody and effector molecule are administered in different compositions and/or are administered at different times and/or by different means.

12. A method of treating cancer in a subject in need thereof, said method comprising administering an allosteric antibody and a prodrug of any one of claims 1-8 to the subject.

13. The method of claim 12, wherein said allosteric antibody and effector molecule are administered in different compositions and/or are administered at different times and/or by different means.
